(19) United States
(12) Patent Application Publication
(10) Pub. No.: US 2010/0158998 A1
(43) Pub. Date: Jun. 24, 2010

(54) FORMULATIONS COMPRISING VITAMIN D OR DERIVATIVES THEREOF

(76) Inventors: Michael Fox, Tel Aviv (IL); Leon Shakib, Holon (IL)

Correspondence Address: KENYON & KENYON LLP, ONE BROADWAY, NEW YORK, NY, 10004, US

(21) Appl. No.: 12/646,619

(22) Filed: Dec. 23, 2009

Related U.S. Application Data
(60) Provisional application No. 61/140,345, filed on Dec. 23, 2008.

(51) Int. Cl. A61K 31/59; A61K 9/48; A61K 9/20; A61K 9/14; A61P 3/14; B05D 1/36; A61K 31/675; A61K 31/66; A61K 31/593

(52) U.S. Cl. 424/452; 424/465; 424/490; 424/493; 424/494; 424/497; 427/402; 427/415; 514/89; 514/108; 514/167

(57) ABSTRACT

The present invention provides stable formulations of vitamin D or a derivative thereof, preferably cholecalciferol.

### CROSS-REFERENCE

This application claims benefit of U.S. Provisional Patent Application No. 61/140,345, filed Dec. 23, 2008, the contents of which are incorporated herein in its entirety by reference.

### FIELD OF THE INVENTION

The invention relates to formulations of vitamin D or derivatives thereof, preferably cholecalciferol, and processes for preparing the same. In particular, the present invention provides stable formulations of vitamin D or derivatives thereof, preferably cholecalciferol, and processes for preparing the same.

### BACKGROUND OF THE INVENTION

Cholecalciferol is a form of vitamin D, also referred to as vitamin D3. Reportedly, cholecalciferol is used in the correction of calcium and vitamin D deficiency in the elderly. Also, cholecalciferol may be used as an adjunct to specific therapy for osteoporosis, in patients with either established vitamin D and calcium combined deficiencies or in those patients at high risk of needing such therapeutic supplements.

For example, alendronate/cholecalciferol combinations are marketed in the UK as Fosavance®, apparently, for use in the treatment of postmenopausal osteoporosis in patients at risk of vitamin D deficiency.

Alendronate is a active ingredient that is part of a group of drugs referred to as bisphosphonates.

During treatment with bisphosphonates, the early inhibition of bone resorption, apparently, induces a decrease in serum calcium, which occurs within days to weeks of the start of treatment. The serum calcium decrease can persist for many weeks to months following the initiation of treatment and can be prominent in vitamin D-insufficient patients. The hypocalcemic response can occasionally be severe enough to be symptomatic and warrant clinical intervention, particularly in patients with hypoparathyroidism and in cancer patients (see Vasikaran, S. D., Ed., 30 Bisphosphonates: An Overview with Special Reference to Alendronate, Ann. Clin.-4, Biochem. (2001)’38: 608-623). As a result, adequate vitamin D (e.g. cholecalciferol) and calcium intake is recommended for subjects using bisphosphonates. Vitamin D supplementation becomes even more critical when calcium needs are elevated due to the net influx of calcium into bone that occurs as a result of bisphosphonate therapy during effective osteoporosis treatment. Reportedly, adequate vitamin D intake is essential to facilitate intestinal absorption of calcium, plays a critical role in regulating calcium metabolism, and is critically important in the mineralization of the skeleton. The primary biological function of vitamin D is to maintain calcium homeostasis by increasing the intestine's efficiency in absorbing dietary calcium and thereby helping ensure that the amount of calcium absorbed is adequate to maintain blood calcium in the normal range and adequate to maintain skeletal mineralization.

However, cholecalciferol is seen to be very unstable and is especially unstable in the presence of oxygen. It has therefore been difficult to provide formulations of cholecalciferol which are stable. This lack of stability may often be detected as a drop in the level of cholecalciferol in a formulation measured using a cholecalciferol assay.

Cholecalciferol, formulations containing cholecalciferol, and process for their preparation have been known since the 1950's.

More recently, WO03/059358 describes oil compositions containing an oil and 25-hydroxy vitamin D3 in which the pharmaceutical active ingredient is dissolved in an oil.

U.S. Pat. No. 4,997,824 describes soft gelatine capsules containing cholecalciferol derivatives in combination with other active ingredients.

It would therefore be highly desirable to provide stable pharmaceutical formulations comprising cholecalciferol. In particular, it would be highly desirable to provide stable solid pharmaceutical formulations comprising cholecalciferol.

### BRIEF DESCRIPTION OF THE DRAWINGS

FIG. 1 illustrates a cross section of the pharmaceutical delivery system in accordance with the present invention when acacia gum was used as an emulsifier;

FIG. 2 illustrates a cross section of the pharmaceutical delivery system in accordance with the present invention when copovidone was used as an emulsifier;

FIG. 3 provide a closer view of a cross section of the pharmaceutical delivery system in accordance with the present invention when acacia gum was used as an emulsifier;

FIG. 4 provide a closer view of a cross section of the pharmaceutical delivery system in accordance with the present invention when copovidone was used as an emulsifier;

FIG. 5 illustrates an outside view of the pharmaceutical delivery system in accordance with the present invention when acacia gum was used as an emulsifier;

FIG. 6 illustrates an outside view of the pharmaceutical delivery system in accordance with the present invention when copovidone was used as an emulsifier;

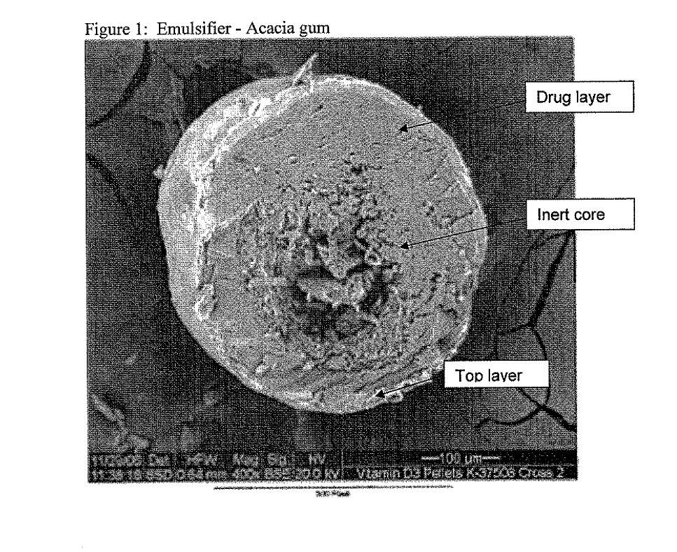

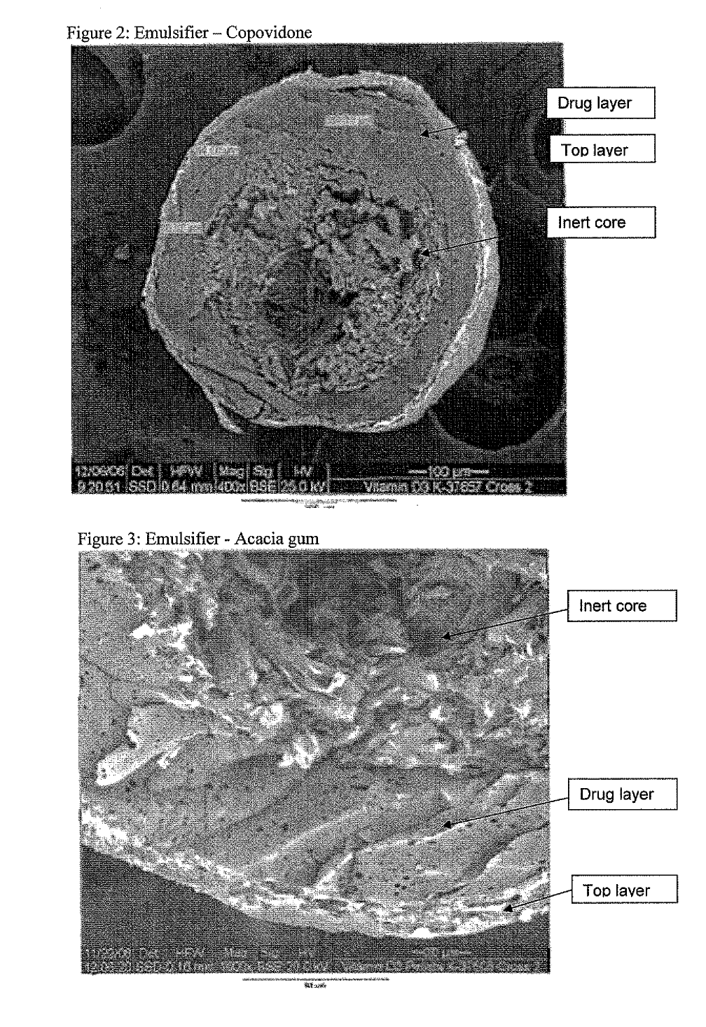

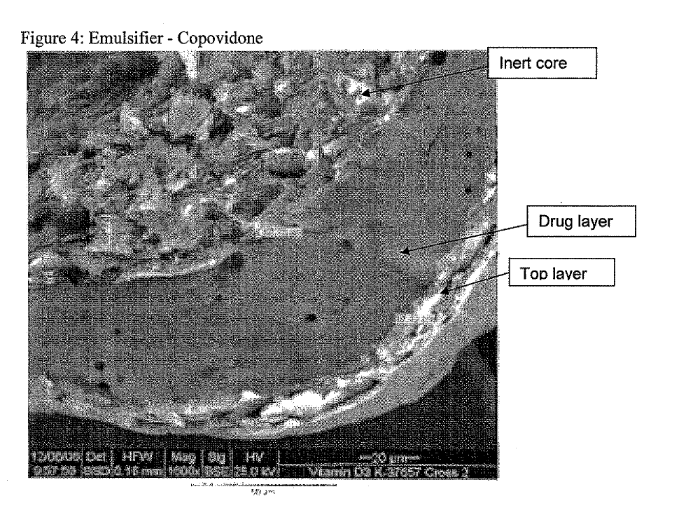

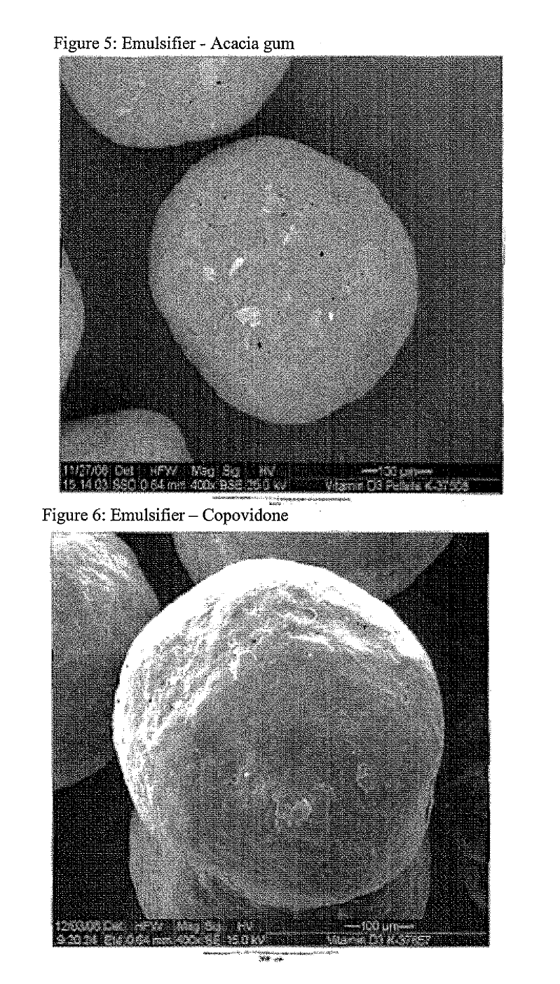

### SUMMARY OF THE INVENTION

The present invention provides a stable pharmaceutical delivery system comprising vitamin D or derivatives thereof, preferably cholecalciferol.

In a first aspect, the present invention provides a pharmaceutical delivery system comprising:
i) an inert core,
an inner layer comprising vitamin D or a derivative thereof, preferably cholecalciferol, an emulsifier and an anti-oxidant, and
iii) an outer protective layer.

In a second aspect, the present invention provides a pharmaceutical composition comprising a pharmaceutical delivery system according to any embodiment of the first aspect of the present invention described herein.

In particular, the pharmaceutical composition further comprises a second active pharmaceutical ingredient. Preferably, the pharmaceutical composition of the present invention further comprises a bisphosphonate such as alendronate, risedronate, ibandronate, zolendronate or a salt thereof.

### DETAILED DESCRIPTION OF THE INVENTION

The present invention provides a stable pharmaceutical grade delivery system comprising vitamin D or derivatives thereof, preferably cholecalciferol.

As used herein “pharmaceutical grade” means produced using validated and well-controlled production procedure.

As used herein “medium chain triglycerides” are medium-chain (6 to 12 carbons) fatty acid esters of glycerol.

As used herein “vitamin D” means vitamin D, isomers or derivatives thereof, or combinations of vitamin D and its derivatives. Preferably the vitamin D derivative is cholecalciferol.

As used herein, the term “bisphosphonate” describes a group of active pharmaceutical ingredients. The meaning of the term is well-known to the person skilled in the art. Preferred bisphosphonates include alendronate, risedronate, ibandronate, zolendronate or salts thereof. A preferred bisphosphonate is alendronate or salts thereof, more preferably alendronate sodium, even more preferably alendronate sodium monohydrate.

As used herein the term “IDD” means impurity and degradation determination. All IDD in the present application refers to impurity and degradation determination of vitamin D or derivatives thereof, preferably cholecalciferol.

In a first aspect, the present invention provides a pharmaceutical delivery system comprising:
i) an inert core,
ii) an inner layer comprising vitamin D or a derivative thereof, preferably cholecalciferol, an emulsifier and an anti-oxidant, and
iii) an outer protective layer.

In a preferred embodiment, the pharmaceutical delivery system is a multiparticulate, such as a pellet, a bead, a sphere etc. Preferably the drug delivery system is in the form of a pellet.

In one embodiment, the inner layer may be applied directly to the inert core. Additionally or alternatively, the outer protective layer may be applied directly to the inner layer.

The inert core provides a substrate to which the inner layer may be applied. The inert core may be a multiparticulate, such as a granule, a pellet, a bead, a beadlet, a microcapsule, a sphere (e.g. a millisphere) etc. Preferably, the inert core is a pellet, a bead, or a sphere. The inert core may be made up of any suitable material, or mixture of materials, such as, for example: sugars, polysaccharides, starches, cellulosic material, inorganics (e.g. glass), and polyols. Preferably, the inert core is made up of glass or sugars. Most preferably, inert core is a sugar sphere or a glass bead.

In particular, the inert core may be formed of microcrystalline cellulose. For example, the inert core may be microcrystalline cellulose pellets. Such microcrystalline cellulose pellets are commercially available and sold under the trade name Cellets™, e.g. Cellets™ 200-350.

Preferably, the inert core is a pellet formed of microcrystalline cellulose, glass or sugars.

The inert core may constitute between about 30 and about 90% (wt/wt) of the pharmaceutical delivery system, preferably between about 30 and about 60% (wt/wt), more preferably between about 30 and about 40% (wt/wt), even more preferably between about 31 and about 36% (wt/wt).

The drug layer is applied using an emulsion comprising vitamin D, derivatives thereof, preferably cholecalciferol, or combinations thereof, and an anti-oxidant. The emulsion may be based on any solvent system suitable for applying the inner layer. Preferably, the solvent system is an oil in water emulsion. In one embodiment, the solvent employed in the emulsion is an organic solvent/water mixture, more specifically an organic solvent in water emulsion. For example, the organic solvent may be medium chain triglycerides. Preferably, when the vitamin D or derivatives is cholecalciferol, the cholecalciferol to solvent ratio is between about 1:20 to about 1:60, more preferably about 1:30. The process for applying the first coating layer to the inert core is described further below.

In a preferred embodiment, the drug delivery system has a maximum diameter of about 600 microns, preferably about 400 to about 600 microns, more preferably about 500 microns or less. Such a maximum diameter may be determined by passing a sample through a 30 mesh sieve.

In a further preferred embodiment, the emulsifier achieves an emulsion having a drop size diameter of about 1 to about 5 microns, preferably about 1 to about 2 microns as measured by optical microscope. Further, the emulsifier is preferably chosen to provide a “dense” layer. A “dense” layer describes a layer which is layered on an inert core in such a way so as to provide an inner layer having a desired density. The desired density is achieved, for example when not less than 85% of the inert core is in a range of about 200 to about 350 microns and the amount of emulsion to be layered on the inert core is 3.5 g emulsion per gram of inert core and the application of the inner layer provides coated pellets with a diameter of about 600 microns or less, preferably about 400 to about 600 microns, more preferably about 500 microns or less.

The emulsifier may be a polaxamer, a polyethylene glycol ethyl ester, a propylene glycol or derivative thereof, acacia, copovidone or combination thereof. Polyvinyl alcohol, polyvinyl alcohol-polyethylene glycol graft copolymer (Kollicoat IR) and gelatine may also be used although for the present invention these are not as preferred as copovidone and acacia. For example, the emulsifier may be acacia or copovidione. The emulsifier may constitute between about 10 and about 30% (wt/wt) of the pharmaceutical delivery system, preferably between about 15 and about 25% (wt/wt), more preferably about 20% (wt/wt).

The antioxidant is employed in the inner layer in order to avoid oxidation of both the active ingredient and the organic solvent e.g. medium chain triglycerides. The antioxidant may be selected from tocopherol (e.g. alpha-tocopherol), ascorbic acid, sodium ascorbate, butylated hydroxyanisole, butylated hydroxytoluene and combination thereof. For example, the anti-oxidant may be butylated hydroxytoluene.

The anti-oxidant may constitute between about 0.1% (wt/wt) and about 2% (wt/wt) based on the weight of the pharmaceutical delivery system, preferably between about 0.4% (wt/wt) and about 1.0% (wt/wt), more preferably about 0.4% (wt/wt) or about 0.9% (wt/wt). Yet more preferably, the anti-oxidant is in constant ratio to cholecalciferol, e.g. cholecalciferol to anti-oxidant ratio is between about 1:1 to about 1:10, and most preferably about 1:4.

The inner layer may also further comprise an additional film former. The film former is capable of forming a solution/dispersion/emulsion and when dried is used as a robust layer. For example, the film former may be selected from sugars, such as lactose, maltose, isomalt, sucrose, starch, xylitol mannitol and combination thereof. In particular, the additional film former may be sucrose.

The additional film former may constitute between about 5 and about 30% (wt/wt) of the pharmaceutical delivery system, preferably between about 10 and about 25% (wt/wt), more preferably between about 10 and about 20% (wt/wt).

If the inner layer comprises an additional film former, the emulsion employed during the application of the inner layer described above further comprises a film former.

The outer protective layer may be any type of coating known in the art suitable for use as a protective layer in a pharmaceutical composition. In particular, a layer, at least one layer or more, which provides adequate protection against oxygen, moisture and light penetration is suitable for use as the protective layer employed in the invention. Preferably, the outer protective layer comprises coating excipients including polyvinyl alcohol (PVA) or hydroxypropyl methyl cellulose (HPMC). For example, the protective layer may be Opadry II 85F18378. Materials with brand name Opadry II and serial 85F are based on PVA which ensure favourable protection against oxygen penetration. Opadry II 85F18378 White has four constituents—titanium dioxide (E171), polyvinylalcohol, macrogol 3350 and talc. Nevertheless, another coating materials based on HPMC and its combinations with lactose, sucrose and other sugars and sugar alcohols may be used. The outer protective layer is preferably in an amount of about 10 to about 30% (wt/wt), more preferably about 20% (wt/wt) based on the weight of the pharmaceutical delivery system. In one embodiment, the outer protective layer is a top coat forming a layer around the outside of the pharmaceutical delivery system.

In another embodiment, the loss of active ingredient from the pharmaceutical delivery system is not more than about 4 percent, preferably about 3 percent or less after storage in a container filled with nitrogen at 40° C. & 75% RH for 3 months, compared to the initial amount at time zero.

In one embodiment of the invention, the pharmaceutical delivery system further comprises a second active pharmaceutical ingredient. Preferably, the pharmaceutical delivery system of the present invention further comprises a bisphosphonate such as alendronate, risedronate, ibandronate, zolendronate or a salt thereof. More preferably, the bisphosphonate is alendronate sodium; in particular alendronate sodium monohydrate is preferred. In one embodiment, the inner layer further comprises a second active pharmaceutical active agent such as described above.

Alternatively, the drug delivery system may contain only a single active pharmaceutical ingredient, i.e. vitamin D or a derivative thereof, preferably cholecalciferol.

In a second aspect, the present invention provides a pharmaceutical composition comprising a pharmaceutical delivery system according to any embodiment of the first aspect of the present invention described herein.

In particular, the pharmaceutical composition further comprises a second active pharmaceutical ingredient. Preferably, the pharmaceutical composition of the present invention further comprises a bisphosphonate such as alendronate, risedronate, ibandronate, zolendronate or a salt thereof. More preferably, the bisphosphonate is alendronate sodium; in particular alendronate sodium monohydrate is preferred. More preferably, the pharmaceutical composition comprises a bisphosphonate as described above and a pharmaceutical delivery system comprising cholecalciferol.

The second active pharmaceutical ingredient may optionally be included as part of the drug delivery system described above, e.g. as part of the inner layer described above. Alternatively and preferably, the second active pharmaceutical ingredient may be included in a part of the pharmaceutical composition which is not the drug delivery system described above. Consequently, in one embodiment of the invention, the pharmaceutical composition comprises a drug delivery system as described above, a second active pharmaceutical ingredient, and at least one pharmaceutically acceptable excipient. Preferably, the second active pharmaceutical ingredient is a bisphosphonate such as alendronate, risedronate, ibandronate, zolendronate or a salt thereof. More preferably, the bisphosphonate is alendronate or a salt thereof, even more preferably the bisphosphonate is alendronate sodium; in particular alendronate sodium monohydrate is preferred.

In a preferred embodiment, the pharmaceutically acceptable excipient is selected from the list comprising a filler, a glidant or a combination thereof. Preferably, the filler is mannitol, microcrystalline cellulose or a combination thereof. Said glidant is preferably colloidal silicone dioxide.

In a particular preferred embodiment, the pharmaceutical composition comprises a pharmaceutical delivery system as described herein, alendronate, preferably alendronate sodium monohydrate, mannitol, microcrystalline cellulose, colloidal silicone dioxide and a lubricant. Preferably, said lubricant is magnesium stearate.

Any conventional tabletting technique may be employed to prepare the pharmaceutical composition of the present invention such as granulation (wet or dry), and direct compression. However, dry granulation and direct compression are preferred.

In a third aspect, the present invention provides a stable pharmaceutical composition comprising a drug delivery system wherein the drug delivery system comprises:
i) an inert core;
ii) an inner layer comprising vitamin D or a derivative thereof, preferably cholecalciferol, an emulsifier, and an anti-oxidant; and
iii) an outer protective layer.

In a preferred embodiment, the loss of active ingredient is not more than about 5 percent, preferably about 4 percent or less after standard accelerated conditions (40° C. & 75% RH for 3 months) or intermediate test conditions (30° C. & 65% RH for 12 months), compared to the initial amount at Time Zero.

Preferably, a composition of the invention contains a level of total impurities and degradation products of about 4 percent or less, about 2 percent, preferably about 1 percent or less after 6 or 12 months of storage under intermediate test conditions of a temperature of about 30° C. and relative humidity of about 65 percent. More preferably, a composition of the invention contains level of total impurities and degradation products of about 1.5 percent, preferably 1.2 percent or less at Time Zero and/or about 4 percent, preferably 3 percent, more preferably 2 percent, yet more preferably about 1.5 percent after 3 months of storage under accelerated conditions of a temperature of about 40° C. and relative humidity of about 75 percent.

In a preferred embodiment, a composition of the invention contains a level of individual impurity of not more than about 1 percent, preferably about 0.8 percent or less, more preferably about 0.6 percent or less, most preferably about 0.5 percent or less after 3 months of storage under accelerated conditions of a temperature of about 40° C. and relative humidity of about 75 percent.

In another preferred embodiment, the pharmaceutical composition comprises a stable pharmaceutical grade formulated particles, more preferably pellets, of vitamin D or derivatives thereof, preferably cholecalciferol or derivatives thereof.

In a fourth aspect, the present invention provides a process for preparing a pharmaceutical drug delivery system comprising vitamin D or a derivative thereof, preferably cholecalciferol, wherein said process comprises:
i) applying an inner coating layer to an inert core to provide a vitamin D or derivative thereof—coated core, wherein the inner coating layer comprises vitamin D or a derivative thereof, preferably cholecalciferol, an emulsifier and an anti-oxidant; and
ii) applying an outer protective layer to the resulting vitamin D or derivative thereof—coated core.

The inert core, emulsifier and anti-oxidant are described above. Suitable protective layers are also described above.

In the coating process of step i), the inert core may be coated with the inner coating layer in a number of ways suitable for applying a coating onto a substrate. A coating process may involve spraying a coating emulsion onto the inert core. In such a spraying process, the coating emulsion can be prepared by emulsifying vitamin D or a derivative thereof, preferably cholecalciferol, and an anti-oxidant in a suitable solvent with an emulsifier. For example, the coating emulsion can be sprayed onto the inert core using a fluid bed coating bottom spray system, such as a Wurster coating system.

The solvent may be any solvent suitable for use in such a coating process. The solvent may be water.

Preferably, the coating emulsion is an emulsion of vitamin D or a derivative thereof, preferably cholecalciferol, in water and an organic solvent in the presence of an anti-oxidant and an emulsifier. More preferably, the coating emulsion is an emulsion of vitamin D or a derivative thereof, preferably cholecalciferol, in water and medium chain triglycerides.

In a preferred embodiment, the coating process of step i) involves:

a) dissolving vitamin D or a derivative thereof, preferably cholecalciferol, and an anti-oxidant in a suitable solvent, preferably under heating, to provide a first solution;

b) dissolving an emulsifier and an additional film foxier in a suitable solvent to provide a second solution;

c) dispersing said first solution in said second solution using a homogenizer to provide a homogenised coating emulsion;

d) coating an inert core with said homogenised coating emulsion to provide a vitamin D or a derivative thereof—coated core.

In step a) above the suitable solvent is preferably medium chain triglycerides.

In step b) above, the suitable solvent is preferably water.

In a preferred embodiment, the coating process of step ii) involves dispersing coating excipients comprising PVA or HPMC, e.g. Opadry II 85F18378, in water to provide a dispersion and coating the vitamin D or a derivative thereof—coated core with said dispersion. The dispersion may be applied in any suitable way, for example by top spray or bottom spray. Preferably, the vitamin D or a derivative thereof—coated core is coated with the dispersion using a Glatt fluid bed coating bottom spray system, such as a Wurster coating system).

In a fifth aspect, the present invention provides a pharmaceutical delivery system comprising an inert core, an inner layer, and an outer protective layer wherein the inner layer comprises vitamin D or a derivative thereof, preferably cholecalciferol, an emulsifier, an anti-oxidant and optionally a bisphosphonate such as alendronate.

In a sixth aspect, the present invention provides a pharmaceutical composition comprising a pharmaceutical delivery system according the fifth aspect of the invention detailed above.

In a seventh aspect, the present invention provides a stable pharmaceutical composition comprising a drug delivery system which comprises an inert core, an inner layer comprising vitamin D or a derivative thereof, preferably cholecalciferol, and, optionally, alendronate, and an outer protective layer. Preferably, the pharmaceutical composition comprises a bisphosphonate as described above, at least one excipient, and a pharmaceutical delivery system as described herein which contains cholecalciferol as the only active pharmaceutical ingredient.

In a preferred embodiment, the at least one excipient is selected from the list comprising a filler, a glidant or a combination thereof. Preferably, the filler is mannitol, microcrystalline cellulose or combination thereof. Said glidant is preferably colloidal silicone dioxide.

For example, the pharmaceutical composition comprises the pharmaceutical delivery system, alendronate sodium monohydrate, mannitol, microcrystalline cellulose, colloidal silicone dioxide and a lubricant. Preferably, said lubricant is magnesium stearate.

In an eighth aspect, the present invention provides a process for preparing a drug delivery system comprising vitamin D or a derivative thereof, preferably cholecalciferol and a bisphosphonate, said process comprising:
i) applying an inner coating layer to an inert core to provide a vitamin D or a derivative thereof/bisphosphonate-coated core, wherein the inner coating layer comprises vitamin D or a derivative thereof, preferably cholecalciferol, bisphosphonate, an emulsifier, and an anti-oxidant; and
ii) applying an outer protective layer to the vitamin D or a derivative thereof/bisphosphonate-coated core.

In an ninth aspect, the present invention provides a process for preparing a pharmaceutical composition comprising vitamin D or a derivative thereof, preferably cholecalciferol, and a bisphosphonate, preferably alendronate, said process comprising dry granulation of a bisphosphonate and at least one excipient (for example, by compaction or slugging, passing the slugs through a mill or an oscillating granulator) to form granules and admixing the granules with a drug delivery system described herein. The admixture may subsequently be sieved and filled into a capsule or be compressed into a tablet. As an alternative to dry granulation, a dry blend of the bisphosphonate and at least one excipient with the drug delivery system comprising vitamin D or a derivative thereof, preferably cholecalciferol, may be compressed directly into a compacted dosage form. In this aspect of the invention, said drug delivery system preferably does not contain a bisphosphonate. Instead, the bisphosphonate is contained in a different part of the pharmaceutical composition.

It has been found to be possible to provide a stable pharmaceutical composition comprising vitamin D or a derivative thereof, preferably cholecalciferol. Preferred pharmaceutical formulation is in a form of a solid dosage form, preferably capsules or tablets.

Having described the invention with reference to certain preferred embodiments, other embodiments will become apparent to one skilled in the art from consideration of the specification. The invention is further defined by reference to the following examples. It will be apparent to those skilled in the art that many modifications, both to materials and methods, may be practiced without departing from the scope of the invention.

### EXAMPLES

### Examples 1-3

| | Example | | | |
|---|---|---|---|---|
| | Ex. 1 | Ex. 2 | Ex. 3 | Ex. 4 |
| Ingredients | Contains, mg/g | | | |
| Cholecalciferol | 1.25 | 1.25 | 2.50 | 1.25 |
| Medium Chain Triglycerides | 38.00 | 38.00 | 76.00 | 38.00 |
| Sucrose | 200.00 | 200.00 | 200.00 | 100.00 |
| Copovidone | — | 200.00 | 200.00 | 100.00 |
| Acacia | 200.00 | — | — | — |
| Microcrystalline Cellulose | 356.00 | 356.00 | 312.00 | 556.00 |
| (Cellets 200-350) | | | | |
| Butylated Hydroxytoluene | 4.75 | 4.75 | 9.50 | 4.75 |
| Opadry II 85F18378 White | 200.00 | 200.00 | 200.00 | 200.00 |

### Example 1

1. 7.41 g of Butylated Hydroxytoluene and 1.95 g of Cholecalciferol were dissolved under N₂ in 59.28 g of hot (about 40° C.) Medium chain triglycerides, using closed vessel.
2. 312 g of Acacia and 312 g of Sucrose were dissolved under N₂ in 1248 g of Purified water, using closed vessel.
3. Solution from step 1 was dispersed under N₂ in the solution from step 2 using a homogenizer.
4. 462.8 g of Microcrystalline Cellulose (Cellets 200-350) were coated with 1617 g of the emulsion from step 3 using Glatt fluid bed coating bottom spray system (Wurster coating).
5. 292.5 g of Opadry II 85F18378 White were dispersed in 900 g of Purified water.
6. Drug coated pellets from step 4 were coated with 1125 g of the dispersion from step 5, using Glatt fluid bed coating bottom spray system (Wurster coating).

### Example 2

1. 7.41 g of Butylated Hydroxytoluene and 1.95 g of Cholecalciferol were dissolved under N₂ in 59.28 g of hot (about 40° C.) Medium chain triglycerides, using closed vessel.
2. 312 g of Copovidone and 312 g of Sucrose were dissolved under N₂ in 1248 g of Purified water, using closed vessel.
3. Solution from step 1 was dispersed under N₂ in the solution from step 2 using a homogenizer.
4. 462.8 g of Microcrystalline Cellulose (Cellets 200-350) were coated with 1617 g of the emulsion from step 3 using Glatt fluid bed coating bottom spray system (Wurster coating).
5. 292.5 g of Opadry II 85F18378 White were dispersed in 900 g of Purified water.
6. Drug coated pellets from step 4 were coated with 1125 g of the dispersion from step 5, using Glatt fluid bed coating bottom spray system (Wurster coating).

### Example 3

1. 783.75 g of Butylated Hydroxytoluene and 206.25 g of Cholecalciferol were dissolved under N₂ in 6270 g of hot (about 40° C.) Medium chain triglycerides, using closed vessel.
2. 16.5 kg of Copovidone and 16.5 g of Sucrose were dissolved under N₂ in 50 kg of Purified water, using closed vessel.
3. Solution from step 1 was dispersed under N₂ in the solution from step 2 using a homogenizer.
4. 23.4 kg of Microcrystalline Cellulose (Cellets 200-350) were coated with 82.9 kg of the emulsion from step 3 using Glatt fluid bed coating bottom spray system (Wurster coating).
5. 18 kg of Opadry II 85F18378 White were dispersed in 54 kg of Purified water.
6. Drug coated pellets from step 4 were coated with 52.6 kg of the dispersion from step 5, using Glatt fluid bed coating bottom spray system (Wurster coating).

### Example 4

1. 326.563 g of Butylated Hydroxytoluene and 85.938 g of Cholecalciferol were dissolved under N₂ in 2613 g of hot (about 40° C.) Medium chain triglycerides, using closed vessel.
2. 6.875 kg of Copovidone and 6.875 kg of Sucrose were dissolved under N₂ in 20.8 kg of Purified water, using closed vessel.
3. Solution from step 1 was dispersed under N₂ in the solution from step 2 using a homogenizer.
4. 34.75 kg of Microcrystalline Cellulose (Cellets 200-350) were coated with 34.16 kg of the emulsion from step 3 using Glatt fluid bed coating bottom spray system (Wurster coating).
5. 15 kg of Opadry II 85F18378 White were dispersed in 45 kg of Purified water.
6. Drug coated pellets from step 4 were coated with 50.0 kg of the dispersion from step 5, using Glatt fluid bed coating bottom spray system (Wurster coating).

### Examples 5-7

The following examples illustrate formulations comprising cholecalciferol in combination with alendronate sodium monohydrate.

Alendronate 70 mg & Cholecalciferol 70 μg/140 μg tablets

| | Example | | |
|---|---|---|---|
| | Ex. 6 | Ex. 6 | Ex. 7 |
| Ingredients | Contains, mg/tablet | | |
| Cholecalciferol Pellets from Ex. 1 | 56.0 | — | — |
| Cholecalciferol Pellets from Ex. 2 | — | 56.0 | — |
| Cholecalciferol Pellets from Ex. 3 | — | — | 56.0 |
| Alendronate Sodium Monohydrate | 81.2 | 81.2 | 81.2 |
| Mannitol | 126.3 | 135.3 | 144.3 |
| Microcrystalline Cellulose | 50.0 | 40.0 | 30.0 |
| Colloidal Silicone Dioxide | 3.0 | 3.0 | 4.0 |
| Magnesium Stearate | 3.5 | 4.5 | 4.5 |
| Theoretical Tablet Weight | 320.0 | 320.0 | 320.0 |

### Example 5

### Alendronate 70 mg & Cholecalciferol 70 μg tablets

1. 243.6 g of Alendronate Sodium Monohydrate, 150 g of Microcrystalline Cellulose and 90 g of Mannitol were mixed together, passed through a 20 Mesh screen and mixed in a Y-blender for 15 minutes.
2. 4.5 g of Magnesium Stearate was passed through a 50 Mesh screen, added to the blend from step 1, and mixed in a Y-blender for 5 minutes.
3. The blend was pressed into slugs using a rotor tablet press with round flat punches.
4. 470.8 g of the slugs were milled through 1 mm screen together with 8.68 g of Colloidal Silicone Dioxide.
5. 155.37 g of Cholecalciferol Pellets from Ex. 1 and 267.18 g of Mannitol were mixed together, passed through an 18 Mesh screen and mixed with 459.73 g of the material from step 4 in a Y-blender for 15 minutes.
6. 5.55 g of Magnesium Stearate was passed through a 50 Mesh screen, added to the blend from step 5, and mixed in a Y-blender for 5 minutes.
7. The final blend from step 6 was pressed into tablets using a rotor tablet press with capsule shaped punches.

### Example 6

### Alendronate 70 mg & Cholecalciferol 70 μg tablets)

1. 324.8 g of Alendronate Sodium Monohydrate, 160 g of Microcrystalline Cellulose and 156 g of Mannitol were mixed together, passed through a 25 Mesh screen and mixed in a Y-blender for 15 minutes.
2. 10 g of Magnesium Stearate was passed through a 50 Mesh screen, added to the blend from step 1, and mixed in a Y-blender for 5 minutes.
3. The blend was pressed into slugs using a rotor tablet press with round flat punches.
4. 561.48 g of the slugs were milled through 1 mm screen together with 10.91 g of Colloidal Silicone Dioxide.
5. 198.8 g of Cholecalciferol Pellets from Ex. 2 and 341.87 g of Mannitol were mixed together, passed through an 18 Mesh screen and mixed with 588.25 g of the material from step 4 in a Y-blender for 15 minutes.
6. 7.1 g of Magnesium Stearate was passed through a 50 Mesh screen, added to the blend from step 5, and mixed in a Y-blender for 5 minutes.
7. The final blend from step 6 was pressed into tablets using a rotor tablet press with capsule shaped punches.

### Example 7

### Alendronate 70 mg & Cholecalciferol 140 μg tablets)

1. 8120 g of Alendronate Sodium Monohydrate and 200 g of Colloidal Silicone Dioxide were mixed in a Y-blender for 5 minutes and passed through 0.8 mm screen using Quadro Comil milling machine.
2. Blend from step 1, 3000 g of Microcrystalline Cellulose and 13000 g of Mannitol were mixed in a Y-blender for 15 minutes.
3. 250 g of Magnesium Stearate was passed through a 50 Mesh screen, added to the blend from step 2, and mixed in a Y-blender for 5 minutes.
4. The blend was pressed into slugs using a rotor tablet press with round flat punches.
5. The slugs were milled through 0.8 mm screen.
6. Milled slugs from step 5, 539 g of Cholecalciferol Pellets from Ex. 3, 200 g of Colloidal Silicone Dioxide and 1491 g of Mannitol were mixed in a Y-blender for 5 minutes, passed through a 20 Mesh screen, returned to Y-blender and mixed for 15 minutes.
7. 200 g of Magnesium Stearate was passed through a 50 Mesh screen, added to the blend from step 6, and mixed in a Y-blender for 5 minutes.
8. The final blend from step 7 was pressed into tablets using a rotor tablet press with capsule shaped punches.

### Example 8

### Cholecalciferol pellets and Alendronate 70 mg & Cholecalciferol 70 μg/140 μg tablets—Stability test results of Cholecalciferol

| | | % Assay of |
|---|---|---|
| Description, test conditions: 40° C., 75% RH | Test Interval | Cholecalciferol |
| Cholecalciferol pellets 2.5 mg/g, Ex. 3 | Time 0 | 101.1 |
| Plasdone S-630 as an emulsifier | 1 Month | 101.7 |
| Packaging - Aluminium bag filled with nitrogen | 2 Month | 99.3 |
| | 3 Month | 99.0 |
| Alendronate 70 mg & Cholecalciferol 70 μg tablets, | Time 0 | 104.7 |
| Ex. 5 | 1 Month | 103.7 |
| Packaging - blister Alu-Alu | 2 Month | 106.2 |
| | 3 Month | 104.2 |
| | 6 Month | 97.5 |
| Alendronate 70 mg & Cholecalciferol 70 μg tablets | Time 0 | 107 |
| Ex. 6 Packaging - blister Alu-Alu | 1 Month | 114.6 |
| | 2 Month | 115.2 |
| | 3 Month | 112.8 |
| | 6 Month | 113.7 |
| Alendronate 70 mg & Cholecalciferol 140 μg tablets | Time 0 | 100.1 |
| Ex. 7 | 1 Month | 101.0 |
| Packaging - blister Alu-Alu | 2 Month | 104.4 |
| | 3 Month | 97.7 |

| Description, test conditions: | | % Assay of | % Total IDD of |
|---|---|---|---|
| 30° C., 65% RH | Test Interval | Cholecalciferol | Cholecalciferol |
| Alendronate 70 mg & | Time 0 | 100.4 | 1.2 |
| Cholecalciferol 140 μg tablets | 6 Month | 99.2 | 0.91 |
| from Ex. 7. | 9 Month | 98.9 | 1.01 |
| Packaging - blister Alu-Alu | 12 Month | 96.7 | 1.01 |
| Description, test conditions: | | % Assay of | % Total IDD of |
| 40° C., 75% RH | Test Interval | Cholecalciferol | Cholecalciferol |
| Alendronate 70 mg & | Time 0 | 100.1 | 1.2 |
| Cholecalciferol 140 μg tablets | 1 Month | 101.0 | 0.89 |
| from Ex. 7. | 2 Month | 104.4 | 1.13 |
| Packaging - blister Alu-Alu | 3 Month | 97.7 | 1.45 |

| | | % Greatest |
|---|---|---|
| Description, test conditions: | | unknown impurity |
| 40° C., 75% RH | Test Interval | of Cholecalciferol |
| Alendronate 70 mg & | Time 0 | 0.4 |
| Cholecalciferol 140 μg tablets | 1 Month | 0.44 |
| from Ex. 7. | 2 Month | 0.43 |
| Packaging - blister Alu-Alu | 3 Month | 0.39 |
| | 6 Month | 0.39 |

| | | % Assay of |
|---|---|---|
| Description, test conditions: 40° C., 75% RH | Test Interval | Cholecalciferol |
| Alendronate 70 mg & Cholecalciferol 140 μg | Time 0 | 100.3 |
| tablets when commercial product (Vitamin D3 | 1 Month | 97.5 |
| 100 SD/S from DSM Nutritional Products, Inc.) | 2 Month | 96.4 |
| comprises Cholecalciferol was used. | 3 Month | 92.0 |
| Packaging - blister Alu-Alu | | |
| | | % Assay of |
| Description, test conditions: 25° C., 65% RH | Test Interval | Cholecalciferol |
| Alendronate 70 mg & Cholecalciferol 140 μg | Time 0 | 100.3 |
| tablets when commercial product (Vitamin D3 | 3 Month | 98.3 |
| 100 SD/S from DSM Nutritional Products, Inc.) | 6 Month | 96.6 |
| comprises Cholecalciferol was used. | 9 Month | 95.5 |
| Packaging - blister Alu-Alu | 12 Month | 93.5 |

Stability was determined by an HPLC method with the following parameters:
Column & Packing: Ace C18, 3μ, 15 cm×4.6 mm
Pre column: Betabasic C18, 2 cm×4 mm
Column Temperature: 27° C.
Detector: UV at 265 nm and 220 nm, 10 mm flow cell path length
   The method should be applied only on HPLC system with dual wavelength detector.
Injection Volume: 50 μL

Diluent: IPA: 0.5% sodium dodecyl sulfate (SDS) solution (60:40 v/v).
Injector Wash Solution: Methanol
Autosampler Temperature: 5° C.±2° C.
Mobile Phase: Eluent A: Acetonitrile
   Eluent B: Purified water

Gradient Time Program:

| Time | Flow | | |
|---|---|---|---|
| (min) | (ml/min) | Eluent A | Eluent B |
| 0 | 1.0 | 50 | 50 |
| 5 | 1.0 | 50 | 50 |
| 8 | 1.0 | 100 | 0 |
| 49 | 1.0 | 100 | 0 |
| 50 | 1.0 | 50 | 50 |
| 55 | 1.0 | 50 | 50 |

Assay and impurity and degradation product were calculated as follows:

For assay:

$$\frac{Total Smp . peak area}{Avg . Std . peak area} \times \frac{Std . conc {{.}_{( mg / ml )}}^{*} \times {Vsmp}_{( ml )}}{L} \times \frac{Avg . tab . {weight}_{( mg )}}{Smp . {Wt}_{( mg )}} \times 100 == \% Assay of Cholecalciferol of the labeled amount$$

$$Total Peak Area = Pre Cholecalciferol Peak Area \times 2 + Cholecalciferol Peak Area$$

For impurities and degradation products:

$$\frac{Imp . peak area}{Avg . IDD Std . peak {area}^{**}} \times \frac{Std . conc {{.}_{( mg / ml )}}^{*} \times {Vsmp}_{( ml )}}{L \times Smp . {Wt}_{( mg )} \times RRf} \times 100 = \% of I D D \\ * Take into account the \% assay and the \% water of the relevant standard \\ ** Avg . I D D std . peak area - at 265 nm or at 220 nm in respective to the I D D at 265 nm or 220 nm .$$

Vsmp—Sample volume
L—Labeled amount: mg Cholecalciferol/mg of pellets
RRf=Relative Response factor. RRf is equal 1.0 for all identified and not identified impurities and degradation products.

### Example 8

### Type of Emulsifier

| | Drops size, |
|---|---|
| Emulsifier | μm |
| Acacia | 2-5 |
| Polyvinyl alcohol-polyethylene glycol | 1-2 |
| graft copolymer (Kollicoat IR) | |
| PVA | 1-2 |
| Gelatin | 1-5 |
| Copovidone (Plasdone S-630) | 1-2 |

Drops size of the emulsion as a result of using the tested polymers, as described in the table above, was determined by measuring the drops, as seen in optical microscope, by using a ruler.

The best results were achieved using acacia gum and copovidone, while copovidone provided more dense, more stable emulsion and easier preparation.

All tested polymers provided oil/water emulsion. However, using some of them (e.g. Kollicoat IR) lead to less dense active ingredient layer and therefore, to bigger pellets. Using big pellets may cause to further tableting problems, so these are not as preferred. Other polymers (e.g. PVA and Gelatin) were found to be less suitable for the Wurster process because of gel formation during drying.

1. A pharmaceutical delivery system comprising:
i) an inert core,
ii) an inner layer comprising vitamin D or a derivative thereof, an emulsifier and an anti-oxidant, and
iii) an outer protective layer.

2. The pharmaceutical delivery system of claim 1, wherein the vitamin D or a derivative thereof is cholecalciferol.

3. The pharmaceutical delivery system of claim 1, wherein the inert core is a multiparticulate comprising a granule, a pellet, a bead, a beadlet, a microcapsule or a sphere (e.g. a millisphere).

4. The pharmaceutical delivery system of claim 1, wherein the inert core is a pellet, a bead, or a sphere.

5. The pharmaceutical delivery system of claim 1, wherein the inert core is made up of any suitable material, or mixture of materials, such as: sugars, polysaccharides, starches, cellulosic material, inorganics (e.g. glass), and polyols.

6. The pharmaceutical delivery system of claim 1, wherein the inert core is a sugar sphere or a glass bead.

7. The pharmaceutical delivery system of claim 1, wherein the inert core is formed of microcrystalline cellulose.

8. The pharmaceutical delivery system of claim 1, wherein the inert core is a pellet formed of microcrystalline cellulose, glass or sugars.

9. The pharmaceutical delivery system of claim 1, wherein the inert core constitute between about 30 and about 90% (wt/wt) of the pharmaceutical delivery system.

10. The pharmaceutical delivery system of claim 1, wherein the inner layer is applied using an emulsion comprising vitamin D or a derivative thereof, and an anti-oxidant.

11. The pharmaceutical delivery system of claim 10, wherein the emulsion is oil in water emulsion.

12. The pharmaceutical delivery system of claim 10, wherein the emulsion is based on an organic solvent/water mixture.

13. The pharmaceutical delivery system of claim 12, wherein the organic solvent is medium chain triglycerides.

14. The pharmaceutical delivery system of claim 12, wherein when the vitamin D derivative is cholecalciferol, the cholecalciferol to solvent ratio is between about 1:20 to about 1:60.

15. The pharmaceutical delivery system of claim 1, wherein the emulsifier achieves an emulsion having a drop size of about 1 to about 5 microns as measured by optical microscope.

16. The pharmaceutical delivery system of claim 3, wherein when the delivery system is in a form of a pellet the maximum diameter is of about 600 microns or less.

17. The pharmaceutical delivery system of claim 1, wherein the emulsifier is selected from the list comprising polaxamer, polyethylene glycol ethyl ester, propylene glycol or derivative thereof, acacia, copovidone or combination thereof.

18. The pharmaceutical delivery system of claim 1, wherein the emulsifier is acacia or copovidione

19. The pharmaceutical delivery system of claim 1, wherein the emulsifier is in an amount of between about 10 and about 30% (wt/wt) of the pharmaceutical delivery system.

20. The pharmaceutical delivery system of claim 1, wherein the antioxidant is selected from tocopherol (e.g. alpha-tocopherol), ascorbic acid, sodium ascorbate, butylated hydroxyanisole, butylated hydroxytoluene and combination thereof.

21. The pharmaceutical delivery system of claim 1, wherein the antioxidant is in an amount of between about 0.1% (wt/wt) and about 2.0% (wt/wt) based on the weight of the pharmaceutical drug delivery system.

22. The pharmaceutical delivery system of claim 1, wherein the vitamin D or derivative thereof to anti-oxidant ratio is between about 1:1 to about 1:10.

23. The pharmaceutical delivery system of claim 1, wherein the inner layer comprises a film former.

24. The pharmaceutical delivery system of claim 23, wherein the film former is selected from sugars, such as lactose, maltose, isomalt, sucrose, starch, xylitol, mannitol and combination thereof.

25. The pharmaceutical delivery system of claim 23, wherein the film former is in an amount of between about 10 and about 30% (wt/wt) of the pharmaceutical drug delivery system.

26. The pharmaceutical delivery system of claim 1, wherein the outer protective layer provides adequate protection against oxygen, moisture and light penetration.

27. The pharmaceutical delivery system of claim 1, wherein the outer protective layer comprises polyvinyl alcohol or hydroxypropyl methylcellulose.

28. The pharmaceutical delivery system of claim 1, wherein the outer protective layer comprises hydroxypropyl methylcellulose in combination with lactose, and sucrose.

29. The pharmaceutical delivery system of claim 1, wherein the outer protective layer is preferably in an amount of about 10 to about 30% (wt/wt) based on the weight of the pharmaceutical delivery system.

30. The pharmaceutical delivery system of claim 1, wherein the outer protective layer comprises OPADRY II 85F18378 containing titanium dioxide (E171), polyvinylalcohol, macrogol 3350 and talc.

31. The pharmaceutical delivery system of claim 1, wherein the outer protective layer is a top coat forming a layer around the outside of the pharmaceutical delivery system.

32. The pharmaceutical delivery system of claim 1, wherein the pharmaceutical delivery system comprises a second active pharmaceutical ingredient.

33. The pharmaceutical delivery system of claim 1, wherein the loss of vitamin D or a derivative thereof from the pharmaceutical delivery system is not more than about 4 percent after storage in a container filled with nitrogen at 40° C. & 75% RH for 3 months, compared to the initial amount at Time Zero.

34. The pharmaceutical delivery system of claim 1, wherein the delivery system is in a pharmaceutical grade.

35. A pharmaceutical composition comprising the pharmaceutical delivery system according to claim 1.

36. The pharmaceutical composition of claim 35, wherein the composition comprises a second active pharmaceutical ingredient and at least one excipient.

37. The pharmaceutical composition of claim 36, wherein the second active pharmaceutical ingredient is a bisphosphonate.

38. The pharmaceutical composition of claim 37, wherein the bisphosphonate is selected from alendronate, risedronate, ibandronate, zolendronate and salt thereof.

39. The pharmaceutical composition of claim 36, wherein the second active pharmaceutical ingredient is alendronate.

40. The pharmaceutical composition of claim 35, wherein the pharmaceutical composition is stable.

41. The pharmaceutical composition of claim 35, wherein the loss of vitamin D or a derivative thereof is not more than about 5 percent, after standard accelerated conditions (40° C. & 75% RH for 3 months) or intermediate test conditions (30° C. & 65% RH for 12 months), compared to the initial amount as was measured at Time Zero.

42. The pharmaceutical composition of claim 35, wherein the composition contains a level of total impurities and degradation products of vitamin D or a derivative thereof of about 4 percent or less after 6 or 12 months of storage under intermediate test conditions of a temperature of about 30° C. and relative humidity of about 65 percent.

43. The pharmaceutical composition of claim 35, wherein the composition contains level of total impurities and degradation products of vitamin D or a derivative thereof of about 1.5 percent or less at Time Zero and/or about 4 percent after 3 months of storage under accelerated conditions of a temperature of about 40° C. and relative humidity of about 75 percent.

44. The pharmaceutical composition of claim 35, wherein the composition of the invention contains a level of individual impurity of vitamin D or a derivative thereof of not more than about 1 percent after 3 months of storage under accelerated conditions of a temperature of about 40° C. and relative humidity of about 75 percent.

45. The pharmaceutical composition of claim 35, wherein the composition comprises a stable pharmaceutical grade formulated particles of vitamin D or a derivative thereof.

46. The pharmaceutical composition of claim 36, wherein the at least one excipient is selected from the list comprising a filler, a glidant or combination thereof.

47. The pharmaceutical composition of claim 46, wherein the filler is mannitol, microcrystalline cellulose or combination thereof.

48. The pharmaceutical composition of claim 46, wherein the glidant is colloidal silicone dioxide.

49. The pharmaceutical composition of claim 35, wherein the composition comprises alendronate, mannitol, microcrystalline cellulose, colloidal silicone dioxide and a lubricant, preferably, said lubricant is magnesium stearate.

50. The pharmaceutical composition of claim 35, wherein the pharmaceutical composition is in a form of a solid dosage form.

51. The pharmaceutical composition of claim 50, wherein the solid dosage form is capsules or tablets.

52. A process for preparing a pharmaceutical drug delivery system of claim 1 comprising vitamin D or a derivative thereof, wherein said process comprises:
i) applying an inner coating layer to an inert core to provide a vitamin D or a derivative thereof—coated core, wherein the inner coating layer comprises vitamin D or a derivative thereof, an emulsifier and an anti-oxidant; and
ii) applying an outer protective layer to the resulting vitamin D or a derivative thereof—coated core.

53. The process of claim 52, wherein the inner layer is applied directly to the inert core.

54. The process of claim 52, wherein the outer protective layer is applied directly to the inner layer.

55. The process of claim 52, wherein the coating process of step i) involves spraying a coating emulsion onto the inert core.

56. The process of claim 55, wherein the coating emulsion is prepared by emulsifying vitamin D or a derivative thereof and an anti-oxidant in a suitable solvent with an emulsifier.

57. The process of claim 55, wherein the coating emulsion is sprayed onto the inert core using a fluid bed coating bottom spray system, such as a Wurster coating system.

58. The process of claim 56, wherein the solvent is water.

59. The process of claim 55, wherein the coating emulsion is an emulsion of vitamin D or a derivative thereof in water and an organic solvent in the presence of an anti-oxidant and an emulsifier.

60. The process of claim 55, wherein the coating emulsion is an emulsion of vitamin D or a derivative thereof in water and medium chain triglycerides.

61. The process of claim 52, wherein the coating process of step i) involves:
a) dissolving vitamin D or a derivative thereof and an anti-oxidant in a suitable solvent to provide a first solution;
b) dissolving an emulsifier and an additional film former in a suitable solvent to provide a second solution;
c) dispersing said first solution in said second solution using a homogenizer to provide a homogenised coating emulsion;
d) coating an inert core with said homogenised coating emulsion to provide a vitamin D or a derivative thereof—coated core.

62. The process of claim 61, wherein in step a) the suitable solvent is medium chain triglycerides.

63. The process of claim 61, wherein in step b) the suitable solvent is water.

64. The process of claim 52, wherein the coating process of step ii) involves dispersing coating excipients comprising polyvinyl alcohol or hydroxypropyl methylcellulose in water to provide a dispersion and coating the vitamin D or a derivative thereof—coated core with said dispersion.

65. The process of claim 52, wherein the vitamin D or a derivative thereof—coated core is coated using a Glatt fluid bed coating bottom spray system, such as a Wurster coating system.

66. A process for preparing the pharmaceutical composition of claim 35, wherein said process comprising dry granulation of a bisphosphonate and at least one excipient to form granules and admixing the granules with the drug delivery system.

67. A process for preparing the pharmaceutical composition of claim 35, wherein said process comprising dry blending of the bisphosphonate and at least one excipient with the drug delivery system.
